(19) United States
(12) Patent Application Publication
(10) Pub. No.: US 2008/0262494 A1
(43) Pub. Date: Oct. 23, 2008

(54) SPINAL TOOL

(75) Inventors: Jesse Moore, Swartz Creek, MI (US); Douglas D. Kave, Byhalia, MS (US)

Correspondence Address: LARSON NEWMAN ABEL POLANSKY & WHITE, LLP, 5914 WEST COURTYARD DRIVE, SUITE 200, AUSTIN, TX, 78730, US

(73) Assignee: WARSAW ORTHOPEDIC, INC., Warsaw, IN (US)

(21) Appl. No.: 11/736,292

(22) Filed: Apr. 17, 2007

(51) Int. Cl. A61B 17/56

(52) U.S. Cl. 606/53

(57) ABSTRACT

A spinal tool is disclosed and can include a first arm and a second arm. A rack can extend from the first arm. Further, a pinion gear can be coupled to the second arm. The pinion gear can engage the rack. The spinal tool can also include a worm gear engaged with the pinion gear.

### FIELD OF THE DISCLOSURE

The present disclosure relates generally to surgical tools. More specifically, the present disclosure relates to surgical tools used to distract vertebra and to compress vertebra.

### BACKGROUND

In human anatomy, the spine is a generally flexible column that can take tensile and compressive loads. The spine also allows bending motion and provides a place of attachment for keels, muscles and ligaments. Generally, the spine is divided into three sections: the cervical spine, the thoracic spine and the lumbar spine. The sections of the spine are made up of individual bones (vertebrae) that are separated from each other by intervertebral discs.

The intervertebral discs function as shock absorbers and as joints. Further, the intervertebral discs can absorb the compressive and tensile loads to which the spinal column may be subjected. At the same time, the intervertebral discs can allow adjacent vertebral bodies to move relative to each other a limited amount, particularly during bending, or flexure, of the spine. Thus, the intervertebral discs are under constant muscular and/or gravitational pressure and generally, the intervertebral discs are the first parts of the lumbar spine to show signs of deterioration.

Facet joint degeneration is also common because the facet joints are in almost constant motion with the spine. In fact, facet joint degeneration and disc degeneration frequently occur together. Generally, although one may be the primary problem while the other is a secondary problem resulting from the altered mechanics of the spine, by the time surgical options are considered, both facet joint degeneration and disc degeneration typically have occurred. For example, the altered mechanics of the facet joints and/or intervertebral disc may cause spinal stenosis, degenerative spondylolisthesis, and degenerative scoliosis.

One surgical procedure for treating these conditions is spinal arthrodesis, i.e., spine fusion, which can be performed anteriorally, posteriorally, and/or laterally. The posterior procedures include in-situ fusion, posterior lateral instrumented fusion, transforaminal lumbar interbody fusion (“TLIF”) and posterior lumbar interbody fusion (“PLIF”). Solidly fusing a spinal segment to eliminate any motion at that level may alleviate the immediate symptoms, but for some patients maintaining motion may be beneficial. It is also known to surgically replace a degenerative disc or facet joint with an artificial disc or an artificial facet joint, respectively.

### BRIEF DESCRIPTION OF THE DRAWINGS

FIG. 1 is a lateral view of a portion of a vertebral column;

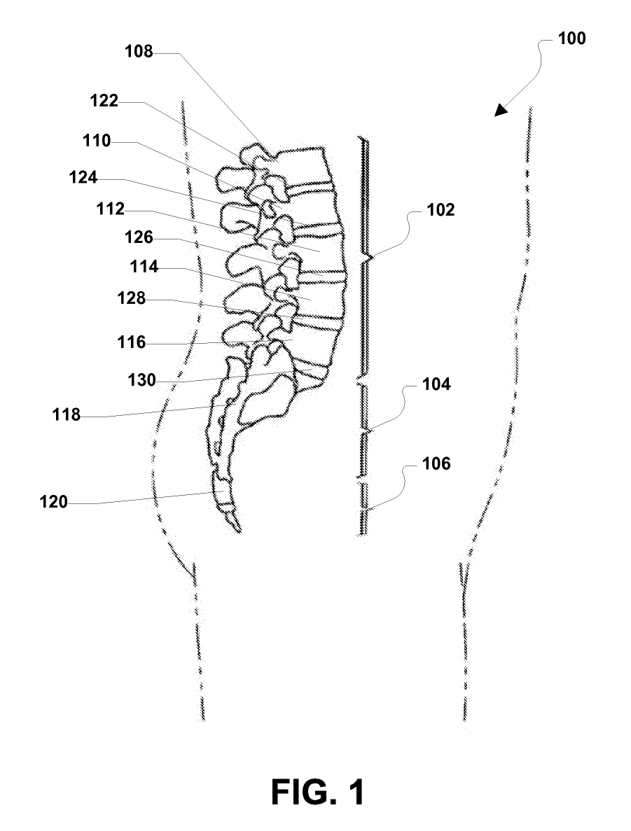

FIG. 2 is a lateral view of a pair of adjacent vertrebrae;

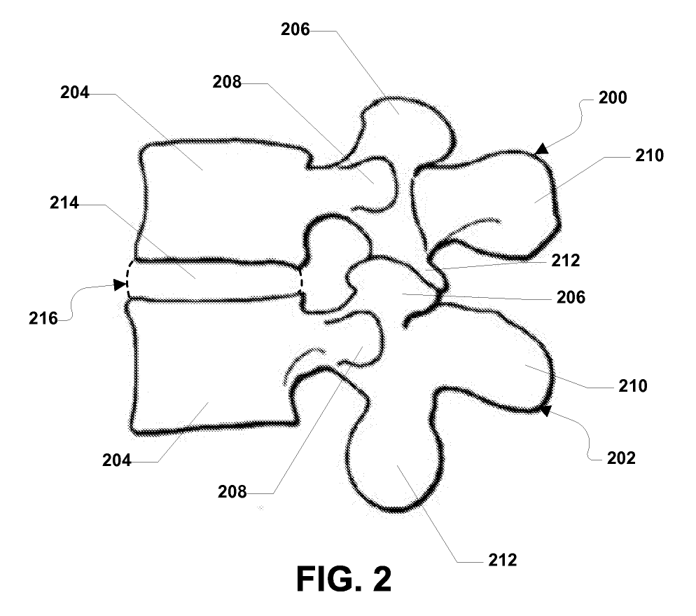

FIG. 3 is a top plan view of a vertebra;

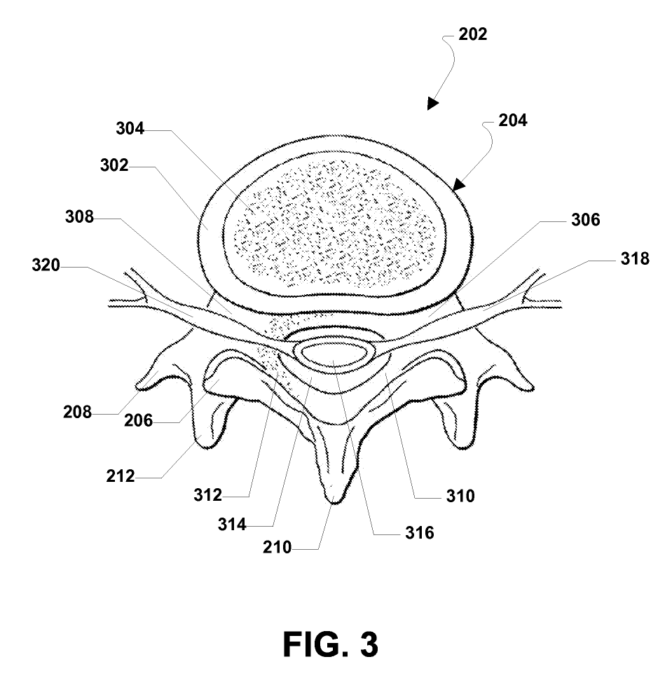

FIG. 4 is a front plan view of a spinal tool;

FIG. 5 is a first lateral plan view of the spinal tool;

FIG. 6 is a second lateral plan view of the spinal tool;

FIG. 7 is a front plan view of the spinal tool with a base of a second arm cross-sectioned;

FIG. 8 is a top plan view of the spinal tool; and

FIG. 9 is a flow chart illustrating one method of using a spinal tool.

### DETAILED DESCRIPTION OF THE DRAWINGS

A spinal tool is disclosed and can include a first arm and a second arm. A rack can extend from the first arm. Further, a pinion gear can be coupled to the second arm. The pinion gear can engage the rack. The spinal tool can also include a worm gear engaged with the pinion gear.

In another embodiment, a spinal tool is disclosed and can include a first arm and a second arm. A rack can extend from the first arm. A pinion gear can be located within the second arm and the pinion gear can engage the rack. The spinal tool can further include a worm gear engaged with the pinion gear. The worm gear can substantially prevent relative motion between the first arm and the second arm while the worm gear is stationary.

In yet another embodiment, a spinal tool is disclosed and can include a rack, at least one gear engaged with the rack, and a worm gear engaged with the at least one gear. The worm gear can substantially prevent the at least one gear from moving relative to the rack while the worm gear is stationary. Further, as the worm gear is rotatable to move the at least one gear relative to the rack.

In still another embodiment, a method of using a surgical tool is disclosed and can include disengaging a pinion gear on the surgical tool from a rack on the surgical tool. Further, the method can include sliding a second arm relative on the surgical tool relative to a first arm and re-engaging the pinion gear with the rack.

In another embodiment, a method of altering a distance between a first vertebra and a second vertebra using a surgical tool is disclosed. The method can include attaching a first arm of the surgical tool to the first vertebra and attaching a second arm of the surgical tool to the second vertebra. The surgical tool can include a rack extending from the first arm and a pinion gear coupled to the second arm and engaged with the rack. Further, the surgical tool can include a worm gear engaged with the pinion gear. The method can also include rotating the worm gear to move the pinion gear along the rack and to move the second arm relative to the first arm.

In yet still another embodiment, a method of enlarging a space within tissue is disclosed and can include engaging a first arm of the surgical tool with a first side of the space and engaging a second arm of the surgical tool with a second side of the space. The surgical tool can include a rack extending from the first arm and a pinion gear coupled to the second arm and engaged with the rack. Further, the surgical tool can include a worm gear engaged with the pinion gear. The method can also include rotating the worm gear to move the pinion gear along the rack and to move the second arm relative to the first arm. As the second arm moves relative to the first arm, the space can enlarge.

### Description of a Spinal Tool

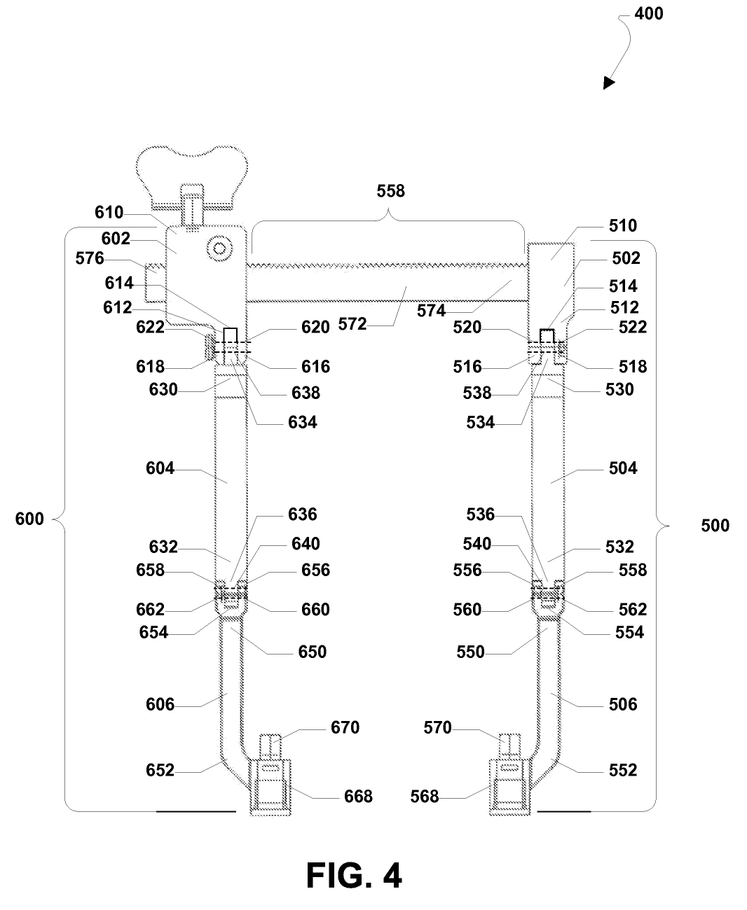

Referring to FIG. 4 through FIG. 8, a spinal tool is shown and is generally designated 400. As shown, the spinal tool 400 can include a first arm 500 and a second arm 600.

In a particular embodiment, the first arm 500 can include a base 502. An intermediate segment 504 can extend from the base 502. Further, a distal segment 506 can extend from the intermediate segment 504. The base 502 of the first arm 500 can have a proximal end 510 and a distal end 512. The distal end 512 can include a distal groove 514 that is flanked by a first distal end portion 516 and a second distal end portion 518. As indicated, the first distal end portion 516 can be formed with a first distal hole 520 and the second distal end portion 581 can be formed with a second distal hole 522.

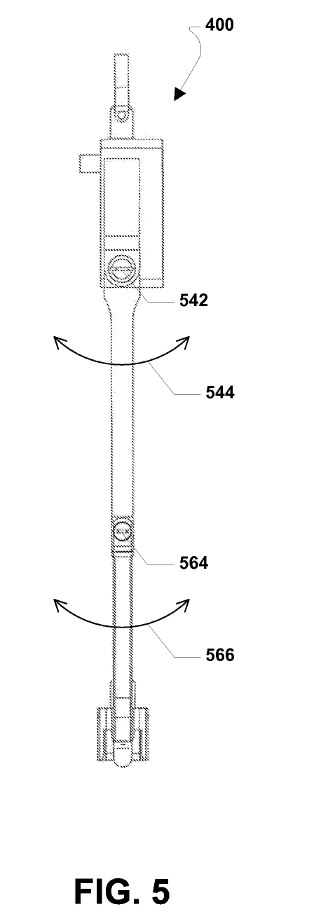

As illustrated in FIG. 4 and FIG. 5, the intermediate segment 504 of the first arm 500 can include a proximal end 530 and a distal end 532. A proximal tongue 534 can extend from the proximal end 520 of the intermediate segment 504. Moreover, a distal tongue 536 can extend from the distal end 532 of the intermediate segment 504. In a particular embodiment, the proximal tongue 534 can be formed with a proximal hole 538 and the distal tongue 536 can be formed with a distal hole 540.

As shown in FIG. 4, the proximal tongue 534 of the intermediate segment 504 can extend into the distal groove 514 of the base 502. A fastener 542 can be inserted through the first distal hole 520 of the base 502, through the proximal hole 538 in the proximal tongue 534 of the intermediate segment 504, and through the second distal hole 522 of the base 502. The fastener 540 can maintain the proximal tongue 534 of the intermediate segment 504 within the distal groove 514 of the base 502. Further, the intermediate segment 504 can rotate with respect to the base 502 around the fastener 540, as indicated by arc 544.

FIG. 4 and FIG. 5 also indicate that the distal segment 506 of the first arm 500 can include a proximal end 550 and a distal end 552. The proximal end 550 of the distal segment 506 can be formed with a proximal groove 554. The proximal groove 554 can be flanked by a first proximal end portion 556 and a second proximal end portion 558. Further, the first proximal end portion 556 of the distal segment 506 can be formed with a first proximal hole 560 and the second proximal end portion 556 of the distal segment 506 can be formed with a second proximal hole 562.

As shown in FIG. 4, the distal tongue 536 of the intermediate segment 504 can extend into the proximal groove 554 of the distal segment 506. A fastener 564 can be inserted through the first proximal hole 560 of the distal segment 506, through the distal hole 540 in the distal tongue 536 of the intermediate segment 504, and through the second proximal hole 562 of the distal segment 506. The fastener 564 can maintain the distal tongue 536 of the intermediate segment 504 within the proximal groove 554 of the distal segment 506. Further, the distal segment 506 can rotate with respect to the intermediate segment 504 around the fastener 564, as indicated by arc 566.

FIG. 4 and FIG. 5 also show that a first collar 568 can extend from, or be attached to, the distal end 552 of the distal segment 506 of the first arm 500. In a particular embodiment, a bone screw 570 can be inserted within, and extend through, the first collar 562.

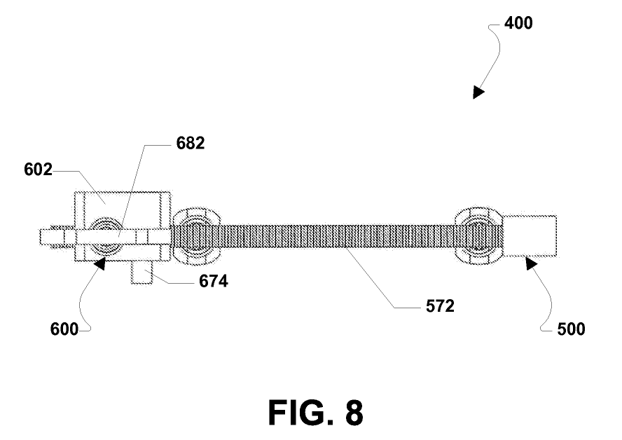

FIG. 4 and FIG. 8 indicates that a rack 572 can extend from the base 502 of the first arm 500. In a particular embodiment, the rack 572 can be substantially straight and can extend substantially perpendicular from the base 502 of the first arm 500. Further, the rack 572 can extend from the base 502 between the proximal end 510 of the base 502 and the distal end 512 of the base. In an alternative embodiment, the rack 572 can be at least partially curved and the rack 572 can extend from the base 502 of the first arm 500 at an angle. Further, in a particular embodiment, the rack 572 can be integrally formed with the base 502 of the first arm 500. Alternatively, the rack 572 can be a separate element that can be press fitted, or otherwise fitted into, a correspondingly shaped hole formed in the base 502 of the first arm 500.

The rack 572 can include a proximal end 574 and a distal end 576. The proximal end 574 of the rack 572 can be attached to the base 502 of the first arm 500. Further, as shown, the rack 572 can be formed with a plurality of teeth 578. As described in detail below, the second arm 600 can move linearly along the rack 572 to enlarge a space within tissue or to reduce a space within tissue. For example, the second arm 600 can move linearly along the rack 572 in order to distract vertebra or compress vertebra.

In a particular embodiment, the second arm 600 can include a base 602. An intermediate segment 604 can extend from the base 602. Further, a distal segment 606 can extend from the intermediate segment 604. The base 602 of the second arm 600 can have a proximal end 610 and a distal end 612. The distal end 612 can include a distal groove 614 that is flanked by a first distal end portion 616 and a second distal end portion 618. As indicated, the first distal end portion 616 can be formed with a first distal hole 620 and the second distal end portion 681 can be formed with a second distal hole 622.

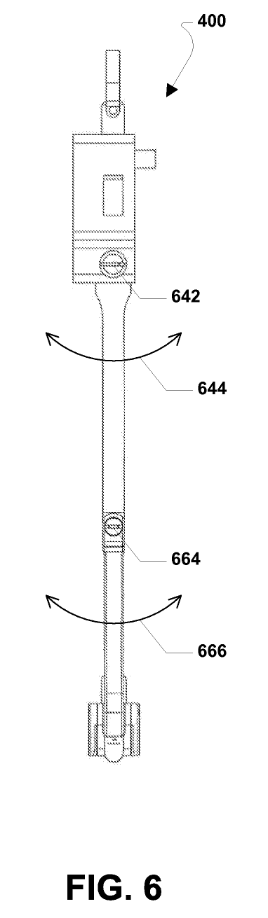

As illustrated in FIG. 4 and FIG. 6, the intermediate segment 604 of the second arm 600 can include a proximal end 630 and a distal end 632. A proximal tongue 634 can extend from the proximal end 620 of the intermediate segment 604. Moreover, a distal tongue 636 can extend from the distal end 632 of the intermediate segment 604. In a particular embodiment, the proximal tongue 634 can be formed with a proximal hole 638 and the distal tongue 636 can be formed with a distal hole 640.

As shown in FIG. 4, the proximal tongue 634 of the intermediate segment 604 can extend into the distal groove 614 of the base 602. A fastener 642 can be inserted through the first distal hole 620 of the base 602, through the proximal hole 638 in the proximal tongue 634 of the intermediate segment 604, and through the second distal hole 622 of the base 602. The fastener 640 can maintain the proximal tongue 634 of the intermediate segment 604 within the distal groove 614 of the base 602. Further, the intermediate segment 604 can rotate with respect to the base 602 around the fastener 640, as indicated by arc 644.

FIG. 4 and FIG. 6 further indicate that the distal segment 606 of the second arm 600 can include a proximal end 650 and a distal end 652. The proximal end 650 of the distal segment 606 can be formed with a proximal groove 654. The proximal groove 654 can be flanked by a first proximal end portion 656 and a second proximal end portion 658. Further, the first proximal end portion 656 of the distal segment 606 can be formed with a first proximal hole 660 and the second proximal end portion 656 of the distal segment 606 can be formed with a second proximal hole 662.

As shown in FIG. 4, the distal tongue 636 of the intermediate segment 604 can extend into the proximal groove 654 of the distal segment 606. A fastener 664 can be inserted through the first proximal hole 660 of the distal segment 606, through the distal hole 640 in the distal tongue 636 of the intermediate segment 604, and through the second proximal hole 662 of the distal segment 606. The fastener 664 can maintain the distal tongue 636 of the intermediate segment 604 within the proximal groove 654 of the distal segment 606. Further, the distal segment 606 can rotate with respect to the intermediate segment 604 around the fastener 664, as indicated by arc 666.

FIG. 4 and FIG. 6 also show that a second collar 668 can extend from, or be attached to, the distal end 652 of the distal segment 606 of the second arm 600. In a particular embodiment, a bone screw 670 can be inserted within, and extend through, the second collar 662.

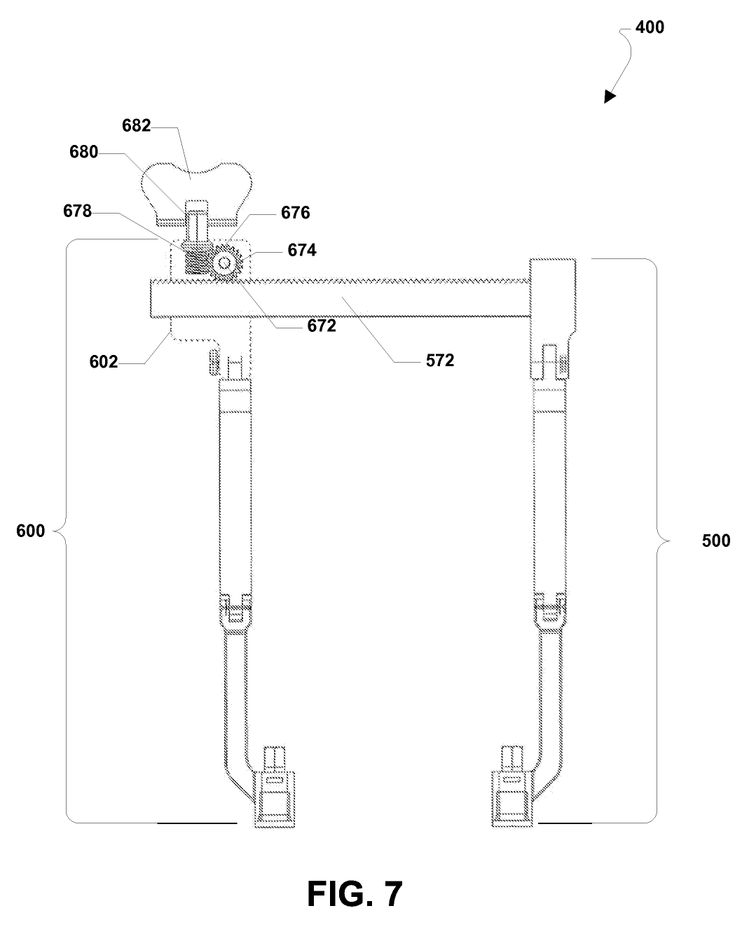

Referring to FIG. 7, a first pinion gear shaft 672 can be disposed within the base 602 of the second arm 600. A second pinion gear shaft 674 can be disposed at least partially around the first pinion gear shaft 672. The second pinion gear shaft 674 can include a pinion gear 676 formed on an end of the second pinion gear shaft 674. A spring (not shown) can be installed around the first pinion gear shaft 672 between the pinion gear 676 and the base 602 of the second arm 600. The spring can bias the pinion gear 676 into engagement with the rack 572 that extends from the first arm 500 of the spinal tool 400. Further, the spring can maintain the pinion gear 676 in engagement with the rack 572. The second shaft 674 can be slid along the first pinion gear shaft 672 toward the base 602 of the second arm 600 in order to slide the pinion gear 676 along the first pinion gear shaft 672 and disengage the pinion gear 676 from the rack 572. As the second shaft 674 is slid along the first pinion gear shaft 672, the spring can be compressed.

FIG. 5 further shows a worm gear 678 engaged with the pinion gear 676. In a particular embodiment, the worm gear can be generally cylindrical with raised ridge forming a helix along the length of the worm gear. The raised ridge can be continuous along the worm gear. Alternatively, the raised ridge can be formed intermittently along the length of the worm gear.

As indicated in FIG. 5, a worm gear shaft 680 can extend from the worm gear 678 through the base 602 of the second arm 600. A handle 682 can be connected to the worm gear shaft 680. As the handle 682 is rotated, the worm gear 678 can rotate with respect to the pinion gear 676. Further, as the worm gear 678 rotates, the pinion gear 676 can also rotate and moves along the rack 572. As the pinion gear 676 moves along the rack 572, the second arm 600 can move relative to the first arm 500. If the worm gear 678 is rotated in a first direction, e.g., clockwise or counterclockwise, the second arm 600 can move towards the first arm 500. Conversely, if the worm gear 678 is rotated in a second direction opposite the first direction, e.g., the other of clockwise or counterclockwise, the second arm 600 can move away from the first arm 500. Accordingly, the spinal tool 400 can be used as a spinal distractor in order to expand a space between two vertebrae or the spinal tool 400 can be used as a spinal compressor in order to collapse a space between two vertebrae.

### Description of a Method of Using a Spinal Tool

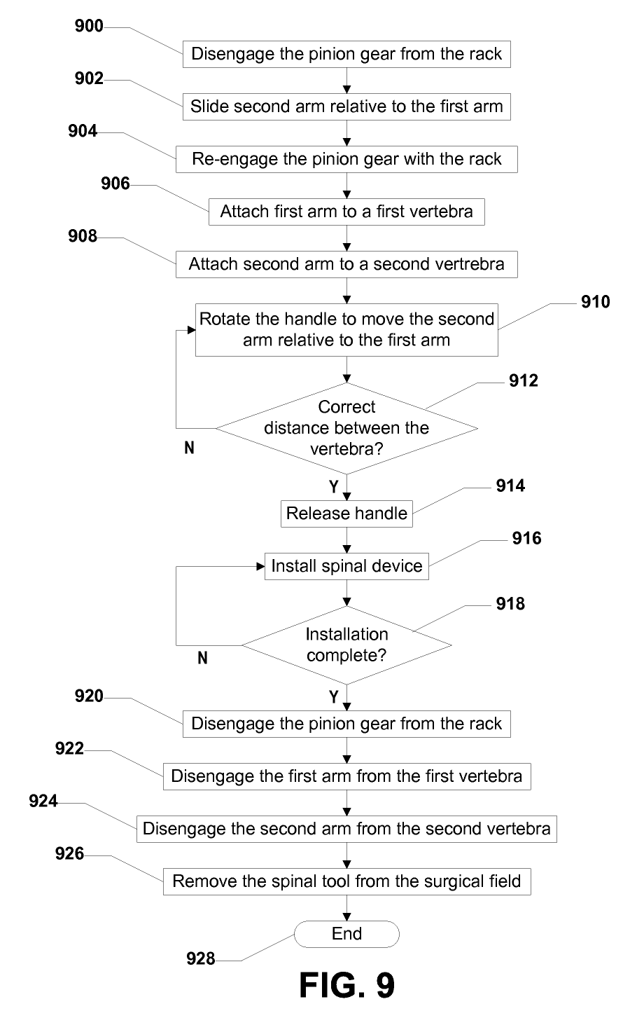

Referring to FIG. 9, a method of using a spinal tool is shown. In a particular embodiment, the spinal tool can be the spinal tool shown and described herein. Further, the spinal tool can include a first arm and a second arm. A rack can extend from the first arm and a pinion gear within the second arm can engage the rack. Also, a worm gear, having a handle connected thereto, can engage the pinion gear. As the handle is rotated, the second arm can move along the rack relative to the first arm.

Commencing at block 900, the pinion gear can be disengaged from the rack. The pinion gear can be disengaged from the rack by sliding a shaft connected to the pinion gear relative to the rack. As such, the pinion gear can be slid off of the rack and disengaged from the rack. At block 902, the second arm can be slid along the rack relative to the first arm. The second arm can be slid toward the first arm or away from the first arm.

Moving to block 904, the pinion gear can be re-engaged with the rack. The pinion gear can be re-engaged with the rack by releasing the shaft and allowing a spring to bias the pinion gear back into position relative to the rack. The shaft can also be rotated in order to properly align the gears on the pinion gear with the rack. At block 906, the first arm can be attached to a first vertebra. Alternatively, the first arm can be attached to a first implant, or a first portion of an implant. The implant can be attached to, or otherwise engaged with, the first vertebra. As such, the first arm can be directly or indirectly attached to, or engaged with, a first vertebra.

Also, at block 908, the second arm can be attached to a second vertebra. Alternatively, the second arm can be attached to a second implant, or a second portion of an implant. The implant can be attached to, or otherwise engaged with, the second vertebra. As such, the second arm can be directly or indirectly attached to, or engaged with, a second vertebra. In a particular embodiment, each arm can be connected to a respective vertebra using a bone screw that can extend through a collar that extends from each arm.

Proceeding to block 908, the handle on the spinal tool can be rotated in order to move the second arm relative to the first arm. In a particular embodiment, rotating the handle in a first direction can move the second arm toward the first arm. Further, rotating the handle in a second direction can move the second arm away from the first arm. At decision step 912, a surgeon can determine whether a correct distance between the vertebra has been reached. If not, the method can return to block 910 and the handle can be rotated further. If a correct distance is reached at block 912, the method can proceed to block 914 and the handle can be released. When the handle is released, the worm gear connected to the handle can prevent the pinion gear from moving relative to the rack. As such, the second arm can be locked in place relative to the first arm and the spinal tool can substantially prevent a distance between the first vertebra and the second vertebra from changing.

At block 916, a spinal device can be installed along the vertebra. In a particular embodiment, the spinal device can be a spinal fixation device, a spinal fusion device, an intervertebral prosthetic disc, a nucleus implant, another spinal device, or a combination thereof. Continuing to decision step 918, it can be determined whether the installation of the spinal device is complete. If not, the method can return to block 916 and the installation of the spinal device can continue. On the other hand, if the installation of the spinal device is complete, the method can continue to block 920 and the pinion gear can be disengaged from the rack, e.g., as described above. In a particular embodiment, disengaging the pinion gear from the rack can relieve any tension on the spinal tool and can allow the spinal tool to be removed from the patient.

Moving to block 922, the first arm of the spinal tool can be disengaged from the first vertebra, e.g., by removing the bone screw connecting the first arm to the first vertebra. Also, at block 924, the second arm of the spinal tool can be disengaged from the second vertebra, e.g., by removing the bone screw connecting the second arm to the second vertebra. Thereafter, at block 926, the spinal tool can be removed from the surgical field. The method can then end at state 928.

### Conclusion

With the configuration of structure described above, the spinal tool provides a device that can be used distract vertebra. Further, the device can be used to compress vertebra. The configuration of the gearing allows the spinal tool to be self-locking and the need for an additional locking mechanism, e.g., a pawl, is obviated. Further, the spinal tool can be used to enlarge a space within other tissue, e.g., the spinal tool can be used a rib spreader or as a retractor. In such a case, the arms of the spinal tool may be modified so that each arm includes a paddle and each paddle can be engaged with a portion of the space. As the handle on the spinal tool is rotated, the arms can move relative to each other and the paddles can cause the space in which the tool is installed to enlarge. When rotation of the handle is ceased, the worm gear can prevent the pinion gear from moving along the rack and can substantially lock the tool and prevent relative motion between the arms - except for any motion caused by deflection of the arms due to external forces placed thereon.

The above-disclosed subject matter is to be considered illustrative, and not restrictive, and the appended claims are intended to cover all such modifications, enhancements, and other embodiments that fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

What is claimed is:

1. A spinal tool, comprising:
a first arm;
a rack extending from the first arm;
a second arm;
a pinion gear coupled to the second arm, wherein the pinion gear engages the rack; and
a worm gear engaging the pinion gear.

2. The spinal tool of claim 1, wherein the worm gear is rotatable in a first direction to translate the second arm toward the first arm.

3. The spinal tool of claim 2, wherein the worm gear is rotatable in a second direction to translate the second arm away from the first arm.

4. The spinal tool of claim 3, wherein the first arm is configured to engage a first vertebra, a vertebral implant, or a combination thereof, and the second arm is configured to engage a second vertebra, a vertebral implant, or a combination thereof, and as the second arm moves toward the first arm a distance between the first vertebra and the second vertebra is reduced.

5. The spinal tool of claim 4, wherein the first arm is configured to engage a first vertebra, a vertebral implant, or a combination thereof, and the second arm is configured to engage a second vertebra, a vertebral implant, or a combination thereof, and as the second arm moves away from the first arm a distance between the first vertebra and the second vertebra is increased.

6. The spinal tool of claim 1, further comprising a worm gear shaft extending from the worm gear and a handle connected to the worm gear shaft, wherein as the handle is rotated, the worm gear rotates.

7. The spinal tool of claim 1, further comprising a first pinion gear shaft, wherein the pinion gear rotates around the first pinion gear shaft.

8. The spinal tool of claim 7, further comprising a second pinion gear shaft around the first pinion gear shaft, wherein the pinion gear is formed on the second pinion gear shaft.

9. The spinal tool of claim 8, wherein the second pinion gear shaft is slidable along the first pinion gear shaft to disengage the pinion gear from the rack.

10. The spinal tool of claim 9, further comprising a spring around the first pinion gear shaft, between a portion of the second arm and the pinion gear, wherein the spring biases the second pinion gear shaft along the first pinion gear shaft to maintain the pinion gear in engagement with the rack.

11. The spinal tool of claim 1, wherein the first arm comprises:
a base segment;
an intermediate segment rotatably coupled to the base segment; and
a distal segment rotatably coupled to the base segment.

12. The spinal tool of claim 11, wherein the first arm further comprises a first collar extending from the distal segment, wherein the first collar is configured to be attached to bony tissue.

13. The spinal tool of claim 1, wherein the second arm comprises:
a base segment;
an intermediate segment rotatably coupled to the base segment; and
a distal segment rotatably coupled to the base segment.

14. The spinal tool of claim 13, wherein the second arm further comprises a first collar extending from the distal segment, wherein the first collar is configured to be attached to bony tissue.

15. A spinal tool, comprising:
a first arm;
a rack extending from the first arm;
a second arm;
a pinion gear within the second arm, wherein the pinion gear engages the rack; and
a worm gear engaging the pinion gear, wherein the worm gear substantially prevents relative motion between the first arm and the second arm while the worm gear is stationary and engaged with the pinion gear.

16. A spinal tool, comprising:
a rack;
at least one gear engaged with the rack; and
a worm gear engaging the at least one gear, wherein the worm gear substantially prevents the at least one gear from moving relative to the rack while the worm gear is stationary and wherein the worm gear is rotatable to move the at least one gear relative to the rack.

17. A method of using a surgical tool, the method comprising:
disengaging a pinion gear on the surgical tool from a rack on the surgical tool;
sliding a second arm relative on the surgical tool relative to a first arm; and
re-engaging the pinion gear with the rack.

18. The method of claim 17, further comprising:
attaching the first arm to a first vertebra;
attaching the second arm to a second vertebra; and
rotating a worm gear engaged with the pinion gear, wherein rotating the worm gear causes the pinion gear to move along the rack and move the second arm relative to the first arm.

19. The method of claim 18, further comprising:
halting the worm gear, wherein worm gear prevents the pinion gear from moving relative to the rack while the worm gear is stationary.

20. The method of claim 19, further comprising:
installing a spinal device relative to the first vertebra and the second vertebra.

21. The method of claim 20, further comprising:
disengaging the pinion gear from the rack.

22. The method of claim 21, further comprising:
disengaging the first arm from the first vertebra;
disengaging the second arm from the second vertebra; and
removing the spinal tool from within a patient.

23. A method of altering a distance between a first vertebra and a second vertebra using a surgical tool, the method comprising:
attaching a first arm of the surgical tool to the first vertebra, wherein the surgical tool includes a rack extending from the first arm;
attaching a second arm of the surgical tool to the second vertebra, wherein the surgical tool includes a pinion gear coupled to the second arm and engaged with the rack and wherein the surgical tool includes a worm gear engaged with the pinion gear; and
rotating the worm gear to move the pinion gear along the rack and to move the second arm relative to the first arm.

24. The method of claim 23, further comprising:
stopping the worm gear to stop the pinion gear from moving along the rack and to substantially prevent the second arm from moving relative to the first arm.

25. A method of enlarging a space within tissue, the method comprising:
engaging a first arm of the surgical tool with a first side of the space, wherein the surgical tool includes a rack extending from the first arm;
engaging a second arm of the surgical tool with a second side of the space, wherein the surgical tool includes a pinion gear coupled to the second arm and engaged with the rack and wherein the surgical tool includes a worm gear engaged with the pinion gear; and
rotating the worm gear to move the pinion gear along the rack and to move the second arm relative to the first arm, wherein the space enlarges as the second arm moves relative to the first arm.

26. The method of claim 25, further comprising:
stopping the worm gear to stop the pinion gear from moving along the rack, wherein the worm gear prevents relative motion between the second arm and the first arm while the worm gear is stationary and substantially prevents the space from collapsing.

27. A method of reducing a space within tissue, the method comprising:
engaging a first arm of the surgical tool with a first side of the space, wherein the surgical tool includes a rack extending from the first arm;
engaging a second arm of the surgical tool with a second side of the space, wherein the surgical tool includes a pinion gear coupled to the second arm and engaged with the rack and wherein the surgical tool includes a worm gear engaged with the pinion gear; and
rotating the worm gear to move the pinion gear along the rack and to move the second arm relative to the first arm, wherein the space reduces as the second arm moves relative to the first arm.

28. The method of claim 27, further comprising:
stopping the worm gear to stop the pinion gear from moving along the rack, wherein the worm gear prevents relative motion between the second arm and the first arm while the worm gear is stationary and substantially prevents the space from enlarging.
